(19) United States
(12) Patent Application Publication
(10) Pub. No.: US 2012/0059378 A1
(43) Pub. Date: Mar. 8, 2012

(54) Efficient Sculpting System

(76) Inventor: James David Farrell, Cincinnati, OH (US)

(21) Appl. No.: 12/954,536

(22) Filed: Nov. 24, 2010

Related U.S. Application Data
(60) Provisional application No. 61/264,590, filed on Nov. 25, 2009.

(51) Int. Cl. A61B 17/16

(52) U.S. Cl. 606/80

(57) ABSTRACT

A system and method to optimize the material removal rate of a tool in a safe and geometrically precise manner, to facilitate the application of smooth contact forces and to sense tool contact forces for rapidly providing power regulation safeguards against tool inadvertently intruding into forbidden regions, for verifying and correlating physically extracted material against the virtual model, and for detecting and mitigating drill walking.

### CROSS-REFERENCE TO RELATED APPLICATIONS

This application claims the benefit of provisional patent application Ser. No. 61/264,590 filed 2009 Nov. 25 by the present inventor.

The following is a tabulation of some prior art that presently appears relevant,

### U.S. Patents

| Patent Number | Kind Code | Issue Date | Patentee |
|---|---|---|---|
| 6,757,582 | B2 | 2004 Jun. 29 | Brisson |
| 7,346,417 | B2 | 2008 Mar. 18 | Luth |

### U.S. Patent Application Publications

| Patent Number | Kind Code | Publication Date | Applicant |
|---|---|---|---|
| 079,898 | B2 | 2005 Mar. 18 | Labadie |

### Non-Patent Literature Documents

Morris D, Sewell C, Barbagli F, Blevins N, Girod S, Salisbury K. Visuohaptic Simulation of Bone Surgery for Training and Evaluation. IEEE Computer Graphics and Applications, Vol. 26, No. 4, November 2006, p 48-57.

### BACKGROUND OF THE INVENTION

Robotic technology has been integrated successfully into the bone surgical process, particularly in operations that involve prosthetic implants for knee replacements. Robotic surgical assistance requires a virtual solid model of the surgical site in order to conduct preoperative planning. A patient-specific solid model with volumetric data is generated by a process of segmenting multiple Computed Axial Tomography (CAT) or Magnetic Resonance Imaging (MRI) scans of imaging data. Typically, the surgeon indicates indirectly the region of the targeted bone material to be resected. For example, the surgeon by overlaying a virtual prosthetic model over the femur (thigh bone) surface has indirectly defined the bounding surfaces of the resectable bone tissue region.

After the physical surgical site is registered with the virtual model, the geometry of the physical surface boundaries of the resectable region is correlated with their virtual solid model counterparts. The registration procedure enables the physical coordinates of the surgical drill pose to be mapped and subsequently monitored by the virtual model navigator (i.e. simulator) of the robotic system. Consequently, the robotic control system can safeguard these surfaces from inadvertent surgical intrusion of the cutting burr by supplying a dynamic resistance to the drill motion as the surgeon guides the tool towards the surface boundary of the resectable region. The benefits of robotic integration are manifested with more accurate and expedient milling and resurfacing operations, reduced inadvertent intrusion into injurious or forbidden regions, reduced surgeon fatigue and less reliance on the adroitness of the surgeon.

In particular, in hard tissue surgery such as bone resection operations, adopted robotic systems serve as intelligent tools for the surgeon to maneuver rather than as autonomous milling devices that mimic classical NC machines. In contemporary commercial offerings, a cutting tool is mounted on an articulated manipulator and subsequently the robotic system allows the surgeon to position the cutting tool within a preoperatively prescribed osseous region. In addition, the robotic system provides active kinematic resistance to the motion of the tool if the surgeon attempts to intrude into a restricted (i.e. injurious) region. This technique requires, at a minimum, an active five degree of freedom system with servo controlled joint actuators, which presents an intrinsic safety issue since “runaway conditions” or unexpected motion are possible due to a hardware malfunction or a software programming error. The implementation effort and maintenance support of an effective safe control to reduce the risk of injury to humans situated within the working envelope of a robot inherently incurs considerable engineering, manufacturing and liability insurance costs.

As a result, prior art has responded to the safety and economical shortcomings of robotic-assisted surgery by controlling the burr rotational speed (Revolutions Per Minute—RPM) of manually guided surgical drills, whose pose (i.e. three dimensional location and orientation) is tracked by a passive device, in order to prevent the surgeon from entering into an injurious region with a rotating burr. The prior art leverages only position data of the drill burr with respect to preoperatively determined surface boundaries of injurious regions in order to determine whether to reduce the RPM of the drill. Prior art does not consider that the osseous structure may consist of a spectrum of material hardness, which inherently represent diverse levels of potential energy required to remove specific regions of material. In particular, the prior art employs only position data of the burr without regard to the contact force applied by the surgeon or the geometric distribution of the energy removal content of the remaining layers of resectable material to determine the drill RPM. Consequently, the optimal drill RPM, which promotes higher surgical throughput and more responsive reaction to potential injurious region intrusion, cannot be achieved by the prior art of manually manipulated and robotically monitored surgical drills.

On another technical front, sophisticated osseous surgical simulators also employ digital solid models, which were generated by segmentation schemes of CAT or MRI scans of bone tissue. These digital models are comprised of voxels (volumetric pixels), which also are employed in robotic surgeries. These training and surgical rehearsal programs conduct real-time modeling of various cutting burrs with the voxel data to accurately predict bone removal rates, which are dependent mainly on the drill cutting capacity and RPM, material hardness associated with voxel data and applied drill forces.

In particular, the rate of bone removal is proportional to the applied contact force of the drill. For example, if the surgeon trainee applies twice the original contact force in a specific resection procedure with all other cutting conditions remaining constant, the simulator will remove the bone at twice rate as the original procedure. These predictive models for bone removal rates cannot leveraged in prior art to estimate accurate arrival times of drill burrs in surgical operations since burr contact forces are not measured.

### SUMMARY

An exemplary embodiment leverages energy removal content of the virtual or physical model and contact drill burr forces to derive optimal RPM values of the drill burr. Also, an embodiment can employ a mechanical scheme that provides compliance along the axial direction of the drill bit shaft in order to attain very accurate depth cuts during the final stages of the resection process.

An exemplary embodiment leverages the technology of the surgical osseous simulation programs to extend their functionality beyond training and surgical preparation in order to serve as a surgical navigational system. This embodiment involves preoperative planning of patient specific virtual models of the surgical site and well established registration techniques and image guidance methods. However, it leverages bone removal rate simulation and modeling techniques by viewing collectively the voxel data enclosed by the swept volume of cutter path as material removal energy. In addition, the drill burr arrival time along a projected straight-line cutter path to the boundary of an injurious region can be accurately estimated from the burr cutting characteristics and RPM, the swept voxel data and the applied contact force of the burr. This embodiment views the bone removal simulation process differently by deriving the drill RPM which generates a cutter path travel time equal to the deceleration time (or marginally greater) of the drill motor (i.e. time to stably stop the drill RPM). This value represents the optimal RPM of the drill.

Consequently, unlike the prior art, it enhances surgical throughput by optimizing the drill RPM of a drill in a safe and non-disruptive manner by sensing applied drill forces and applying energy principles. It employs an approach of leveraging energy principles to accurately predict drill burr travel times of potential cutter paths to nearby injurious regions during the bone removal process. The calculations associated with each potential cutter path are independent. Consequently, the analysis of each potential cutter path can be addressed by a separate execution thread and separate processor core. The analytical technique to predict an arrival time along a specific path of the drill burr to an injurious region will involve quantifying the burr cutting characteristics, sensing contact forces applied by the surgeon, and discerning the geometric distribution of the energy removal content of the remaining osseous resection material. These bone removal rate techniques, which have successfully been integrated and demonstrated in the surgical simulators, are well published and documented in technical literature.

An exemplary embodiment seeks to maximize the RPM of the cutting drill without intruding into an injurious or forbidden region with a rotating drill. The optimal RPM will must allow the rotation of the drill to be stopped within the time period dictated by the capacity of the drill motor. For example, if it requires 80 ms (milliseconds) to stop the rotation of the drill then an optimal RPM cannot create a situation, in which the drill burr may possibly reach an injurious zone in less than 80 ms. The drill RPM is the only variable that can be altered to guarantee that the travel time of the drill burr equals (or exceeds) the deceleration and settling time of the drill motor. This approach requires that the contact translational forces of the drill be measured at sampling rates in the 250 Hz or higher range.

An embodiment addresses otologic surgical procedures, which represent one of the most demanding surgical applications with respect to its precision requirements, limited footprint of the target site, complex and varied anatomical structures and close proximity to many highly critical and vulnerable anatomical structures. Otologic surgical procedures such as mastoidectomy, acoustic neuroma resection, and cochlear implantation, involve drilling on the temporal bone to access critical anatomy within the middle ear, inner ear, and skull base. In particular, this embodiment offers a viable and cost effective alternative to robotic-assisted bone surgery by enabling a surgeon to manually attain high degree of accuracy in a highly efficient and smooth manner. Notwithstanding, this optimal RPM scheme could be adopted by robotic surgical systems as well.

Otologic surgeons develop and customize a variety of stroking and contact techniques to precisely control bone removal and to reduce the risk of inadvertent drill motion that could injure critical anatomical structures. The bone removal process typically represents a significant component of the surgical time for otologic procedures, which are conducted with a series of burrs (rotary drill heads) that vary in size, shape and surface abrasiveness. Larger burrs are generally employed for coarse bone removal in the early stages of a resection procedure and smaller burrs are required for more precise resection to form the final target anatomical cavity.

The operating principles and innovations of this embodiment for otologic procedures can be readily applied to other less demanding hard tissue surgical procedures. Also, the disclosed innovations can readily be extended to non-surgical applications such as woodworking, sculpturing, etc, particularly if the material (i.e. wood, plaster, etc.) of the work piece has reasonably predictable material hardness properties or the distribution of the material hardness is known. In cases, in which the physical properties of the target material are consistent, (non-voxel based) removal rates can be empirically and readily attained by very simple drilling procedures. Regardless of whether voxel data or empirical methods are employed to determine energy removal content, very precise and complex shapes will be able to be extracted in a nearly optimum manner with any of the disclosed embodiments.

The prior art of surgical resection applications with a manually maneuvered drill achieve inefficient but reasonably safe throughput by commanding suboptimal RPM (revolutions per minute), which represents the highest rotational drill speed that can be stopped before the surgeon inadvertently injures a critical anatomical region with a rotating drill burr. In response, this embodiment serves as an intelligent surgical tool that closely optimizes the RPM of the drill while safeguarding errant penetration in an injurious region with a rotating drill. Since one embodiment involves a drill manually maneuvered by a surgeon, it does not employ an active joint-based motorized robotic system to kinematically restrain the surgeon's attempts to remove bone in restricted regions. In a sense, the rotational speed of the drill emulates the active kinematic constraint of a traditional robotic system but in a safer and more passive manner by slowing the drill burr RPM to provide resistance as the drill approaches a surface boundary of an injurious region.

Prior art does not consider burr cutting characteristics, volumetric distribution of bone density and material hardness and the applied force of the surgeon as criteria to control the RPM of the drill. This embodiment utilizes combinations of the following, a passive position measurement device such as an articulated coordinate measurement machine (ACMM) to derive and monitor the pose of the drill burr in a real-time manner, a virtual simulation (i.e. navigation) program that performs high frequency cutting operations of the burr intersecting with the bone tissue and also interprets a detailed volumetric map of bone densities of the target surgical site, translational contact force sensing of the cutting burr, and a servo motor to precisely control the cutting drill rotational speed with a highly responsive brake or a potentially high braking torque provided by the motor. Other passive measurement devices that could replace or complement the ACMM include well established, commercially available, optical, acoustic and magnetic tracking technologies. Also, other material removal technologies such as laser ablation could be employed without force sensing but with energy content removal schemes to regulate the power (i.e. removal rate), which is analogous to controlling the RPM of the drill. In a non-drilling scenario, the power level required to achieve a removal rate would produce a material removal time that was equal to the time to power down the tool.

The embodiment utilizes a high frequency (i.e. 250 Hz or greater) calculation of the material unit power or power constant, which is the energy rate to remove via milling/drilling one cubic inch of material per minute. The cubic inch of material represents specific material removal energy content. Typically, the amount of unit power is proportional to the material hardness, which is the case for bone tissue and other material such as wood and plaster. This embodiment computes the material removal energy content of a resectable bone segment that is enclosed along a possible candidate volumetric tool cutting path. The swept volume of the candidate tool path contains volumetric bone density and material hardness data, from which material removal energy content can be calculated. The Voxmap-Pointshell algorithm, which facilitates collision and intersection detection and proximity estimations for voxel based models, could be employed to capture the voxel data while the tool cutter simulates approaching an injurious surface along a candidate path. That is, the energy calculation process leverages the patient's specific virtual 3D model of the target bone site, which was generated by CAT or MRI scans. As one possible means to determine cutter path energy content, the simulator could emulate a candidate drilling process with the active drill burr but with advantageously high RPM and contact forces to expedite the process. The product of the travel time of the burr and the energy removal rate of the cutter estimates the energy removal content along each of the prospective cutter paths.

The simulator could terminate the cutting process after predetermined energy content was accumulated. In particular, the derivation of the energy content of the entire candidate cutting path to a surface boundary will not be necessary if preliminary calculations indicate sufficient energy content such that the tool can be operated at a specified maximum RPM (material removal rate) for a previously established safe period of time. For example, if the surgeon sets the maximum drill speed at 20,000 RPM then the program can determine from the established deceleration capabilities of the drill motor that 300 ms is sufficient to stop the drill under any circumstances. Consequently, while the controller simulates the tool traveling towards its perspective path if its accumulated travel time exceeds 300 ms then no further tool path travel simulation is required.

Any RPM value, which is greater than the lowest derived RPM for all prospective cutter paths, will generate a travel time less than the deceleration time. Consequently, the lowest RPM value derived from all of the prospective paths will serve as the optimal RPM.

The contact force sensing permits the controller to react much more responsively to forces applied by the surgeon than a position only sensing scheme employed in prior art. A three degree of freedom (DOF) translational force sensing capability enables the controller to apply the proper RPM to create the optimal cutting rates in response to the contact forces applied by the surgeon. In particular, a force sensing capability enables the control system to adjust the RPM rapidly in situations where the burr is close to the surface of an injurious region. A position only sensing scheme must deduce velocity and accelerations from a history of positions in order to determine the proper action, which relies on very accurate measurements in order not to generate large derivative errors. A position only sensing scheme has an associated excessive and unresponsive time lag and subsequently lower bandwidth RPM control.

The burr contact translational feedback forces will need to be filtered in order to attenuate high frequency forces caused by the rotation of the drill removing the bone or other material. However, low bandwidth filtering parameters must be judiciously applied in order not to create an unresponsive phase lag. Also, a motor that provides torque feedback of the drill burr would provide additional data about the cutting power of the tool and would help infer dynamically the cutting resistance of the material (i.e. power constant of the material) during the surgical process.

A three DOF translational force sensing capability helps reconfirm or possibly adjust the computed geometric shape of the remaining workpiece material tracked by the virtual model simulation program. Also, three DOF force sensing permits the controller to monitor free motion (drill burr is not in contact with material) of the drill. As a safety feature, the controller could sufficiently reduce the rotational speed of the drill or stop the drill from rotating if insufficient contact forces are detected and subsequently re-enable and resume the drill speed only after the contact of the burr with the material is detected. With this safeguard active, the surgeon, in practice, would lightly press the drill burr on the bone to reactivate the drilling process. The position only sensing of prior art does not possess the volumetric measuring accuracy to differentiate whether the surgeon is pressing on the bone tissue. This drill enabling protocol would minimize the frequency of occurrences and the collateral damage of inadvertent motion of the free motion drill. On the other hand, the controller could stop the rotation of the drill if excessive applied forces are detected. Again, the force limits could be setup by the surgeon as a safety feature that guarantees light and delicate forces near critical anatomical regions. Also, the three DOF translational force sensing capability has an auxiliary benefit of reducing the number of prospective drill cutter paths that must be analyzed to derive an optimal RPM.

Moreover, an important benefit of a full six DOF force sensing capability (translational and rotational forces are measured) is the ability for the control to detect and mitigate very responsively the detrimental effects of drill walking by systematically reducing the drill RPM. Drill walking occurs if the drill catches a bone fragment and tends to run away from or towards a surgeon's hand. A six DOF force sensing can determine that the drill is being handled properly the surgeon by analyzing its moment readings. With no force sensing capability, prior art controllers cannot distinguish drill motion that was caused by the surgeon intentionally stroking the bone or by the tool walking from the tangential contact forces of the rotating burr.

### Advantages

This embodiment applies virtually optimal drill rotational speed (RPM) under the condition that it can stop the drill rotation before the surgeon errantly intrudes into a critical anatomical structure. It will reduce the operational time and attendant fatigue of the surgeon. Drill walking can be detected early by the controller and subsequently its effect can be mitigated by reducing the RPM systematically. Force sensing will enable control strategies that transition the RPM to enhance operational ease with minimal reduction of productivity. The incorporation of force sensing and energy principles also will enable the control to emulate more precisely (than position only control) the active kinematic constraint of a surgical robotic system but in a passive manner by algorithmically reducing the RPM of the drill to provide dynamic resistance as the tool approaches an injurious region. This resistance represents a viable cue to indicate the drill burr lies within close proximity of an injurious region. The combination of the force sensing and drill feed rate support a simple procedure to empirically derive the material energy content of materials such as plaster, wood, etc. The ACMM embodiment affords the opportunity to offer a low cost and portable alternative to a robotic implementation.

### DESCRIPTION OF DRAWINGS

In the drawings, closely related figures have the same number but a distinct alphabetic suffix.

FIG. 1 is an overview of one of the possible embodiments, in which the passive tracking device is an articulated coordinate measurement machine (ACCM). It continuously monitors the surgical drill pose.

FIG. 2 is a one possible design of an exterior drill casing, which is rigidly and proximally attached to an inside drill structure.

FIG. 3 illustrates the block diagrams of the embodiment of control methods that are executed only once during the preoperative stages.

FIG. 4A illustrates the block diagrams of the embodiment of the iterative control methods that are performed during the intra-operative stages.

FIG. 4B illustrates the block diagrams of the embodiment of the iterative force input and low pass filter methods that are performed on a dedicated execution thread during the preoperative and intra-operative stages.

FIG. 5 illustrates a prospective cutter path, whose start and end points are respectively the current location of the drill burr and the closest point of an injurious region to the drill burr.

### DETAILED DESCRIPTION

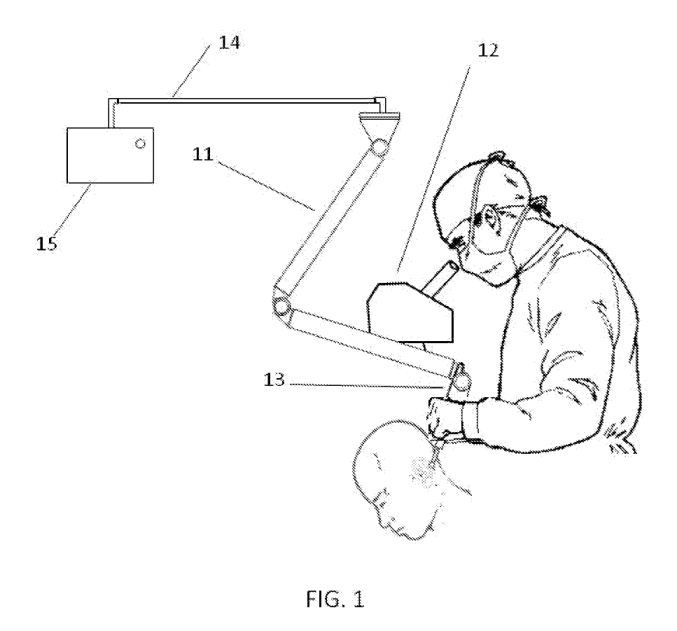

FIG. 1 illustrates an articulated coordinate measurement machine (ACCM) 11 as the tracking device in lieu of other candidates such as infrared optical trackers with three active infrared light emitting markers on the tool. The ACCM 11 consists of a configuration of seven joints with high resolution encoders, which permits the location and orientation of the drill burr to be tracked. The ACCM 11 affords the opportunity to instill high accuracy in the passive tracking system. Well established kinematic calibration techniques, which involve regression analysis and joint mapping techniques, permit volumetric measurement accuracies of 0.0005″. Thermal and gravity models may be employed to compensate for the thermal expansion and distortion and load deflection of the ACCM structure. However, an ACMM structure consisting of composite materials, which are extremely stiff and have very low thermal expansion coefficients, may avoid thermal and load deflection compensation since the structure will be subjected to relatively minor loading conditions and narrow temperature ranges. The design of the ACMM includes a tool interface to mount the drill and an interior passageway to accommodate an internal wiring harness to service the drill, force sensor, joint feedback, lighting, visual cues, etc.

The kinematic redundancy (more than six joints) of the ACMM, allows it to be configured to be less obtrusive, more maneuverable to avoid obstacles, and to have lower apparent tool inertia. For each degree of freedom exceeding six, a joint must be physically locked in order to prevent the arm from internally collapsing within its null space configuration. Any combination of joints can incorporate locks but only one of the joints can be locked at a time. Furthermore, the ACMM embodiment can accommodate a configurable counterbalance scheme to minimize the surgeon's effort with supporting the drill during the surgical operation.

In this embodiment, the surgeon employs a high resolution digital microscope 12, in which transparent cueing images such as color coded material power constants, depths, travel times, etc. of the remaining material are overlaid on the real time image of the surgical site. This augmented reality approach allows the surgeon to remain focused on his surgical field of view. A display of the updated virtual model could be inserted into the viewing area, which does not block the surgeon's field of view. Otherwise, a separate display monitor of the updated virtual model with surgical cueing data can be employed but the surgeon will be required to look away from the surgical site and correlate the simulation data with the physical site. A separate screen is more time consuming and prone to error.

FIG. 1 includes the surgical drill 13 mounted on the distal end of the ACCM. The drill provides contact force sensing capabilities by measuring the strain of its load bearing structure. The strain reflects the contact forces of the cutting burr. A cable 14, which encloses signal and power wiring, is guided through the interior passageway of the ACCM in order for the controller 15 to receive joint position and possibly velocity feedback, burr contact forces, and drill motor RPM feedback and to command the appropriate drill RPM and update the real-time image of the digital microscope with surgical cues via augmented reality techniques. The real-time controller represents a subset of the technology employed for robotic manipulators since no actuated joints are incorporated into the ACCM. In this embodiment the controller employs state of the art multicore processor technology in order to support parallel processing of real-time control techniques and the concomitant surgical image guidance (i.e. simulation) programs.

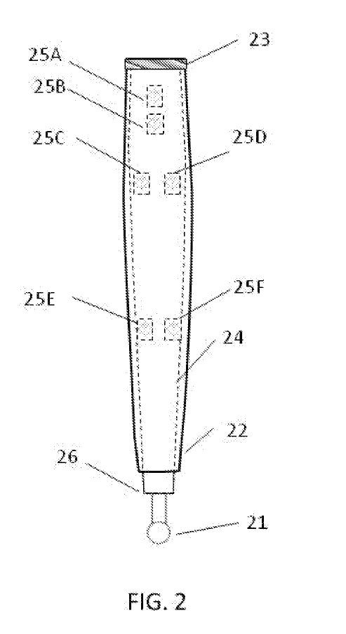

FIG. 2 illustrates a drill with a burr 21 with a stiff outer shell or casing 22 that is mounted rigidly only at the proximal end 23 of the internal load bearing drill structure 24. The outer shell structure shields the force strain gauges of the internal structure. This design enables all of the forces exerted by the surgeon to be transferred through this rigid mounting. As a result, this arrangement provides considerable latitude on the placement of the strain gauges 25A, 25B, 25C, 25D, 25E, and 25F. Only six strain gauges with arbitrary locations are depicted for illustrative purposes. The actual number of strain gauges and their placements and orientations to capture orthogonal translational and rotational forces are based on a well-published and proven Wheatstone bridge circuit design techniques, in which a strain gauge serves as one of the resistors, and attendant conditioning electronics.

One derivative of this embodiment captures three orthogonal translational forces. The axial load from burr contact generates a proportional axial compression/tension strain, which can be measured accurately with two sets of paired gauges connected to a full Wheatstone bridge circuit. The lateral loads (i.e. translational forces normal to the drill bit shaft) from the burr contact create shear and moment strains along the internal circular casing. One side of the casing will be in tension (i.e. longer length) and the opposite side (i.e. 180 degree) will be in compression (i.e. shorter length) due to a lateral load at the burr. The moment strains can be measured to deduce the lateral contact force at the burr. With this gauge configuration, the length of the tool bit must be known to derive the force. However, if a second set of strain gauges that measures moment strain is placed at a known axial offset distance from the first set then the lateral force of the burr can be derived without prior knowledge of the drill bit length. Moreover, the contact length or drill bit length should be able to be inferred by the two distinct sets of moment readings. This additional force sensing adds another safety check that the calibrated length of the tool bit is correct. Also, the controller will be able to detect if the burr rather than the drill bit shaft is making contact with the bone if the drill bit shaft length had been previously established.

There will be cross coupling of strain measurements that are caused by bending moments from the lateral forces and by the axial force of the burr. For example, in the case of a load bearing interior casing with a hollow circular cross section, a pure axial load can be deduced only after the bending moment strain has been resolved. Pure bending moments can be derived from two strain gauges that lie on the same annular shell but at 180 degrees from each other. Any difference in measurement can be attributed to pure bending strain. This bending strain must be extrapolated and recalculated at the location where the gauges measure axial strain in order to mask the moment strain effect from the axial load strain. Two sets of strain gauge pairs at 90 degree offsets along an annular shell will be required to detect completely the lateral load of the burr. Each one within a pair will be offset by 180 degrees. A number of 90 degree offset configurations could be added axially along the structure with angular offsets from the neighboring one.

In order to achieve very high fidelity with respect to discerning steady state contact forces from extraneous and transient forces caused by drill rotations, dynamic filtering strategies and compromises must be realized. Fortunately, the wide disparity between the bandwidth of contact forces and the frequency of the rotationally induced forces will be very conducive to filtering techniques. The human muscles typically perform with a force bandwidth in the approximate range of 2.0 Hz to 10.0 Hz and consequently the applied burr contact forces will exhibit the same responsive behavior. On the other hand, the drill typically operates at minimum of 1,000 RPM, which with multi-fluted burrs increases the force frequency proportionally (i.e. a double fluted burr would generate a 2,000 Hz frequency force signal). In general, rotational speeds for surgical drills during an osseous operation range from 2,000 RPM to 80,000 RPM.)

The active bandwidth of the filter must balance the benefit of decreasing the perceived magnitude of the rotational forces against the shortcoming of increasing the phase lag (i.e. time delay—degree of stale data) of relevant applied contact forces. If the filter bandwidth is decreased, the phase lag of the valid contact data increases but the magnitude of the high frequency rotational forces decreases. Opportunely, dynamic filtering strategies will be able to exploit a monitoring phenomenon that as the drill RPM decreases, the more phase lag can be tolerated by the control process since the removal rate of the burr is reduced. During low RPM rates the filter bandwidth can be decreased to virtually negate the effects of rotational forces.

Consequently, well established low pass filtering techniques, which are applied to the strain gauge reading, will attenuate significantly the effects of the high frequency cutting forces on the measurement derivations without causing an appreciable time lag with respect to the response of the system reacting to the low frequency forces applied by the surgeon. The time constants of the low pass filters can be adjusted based on the RPM of the drill, feed rate of the surgeon and the bone density in the immediate region of the burr in order to achieve a nearly optimal frequency response of the low bandwidth forces applied by the surgeon.

The design of the outer casing structure does not require that the location of the force sensors be situated between the burr holder 26 and surgeon's hand, which would be a necessity for the load bearing, single casing design of prior art. The strain gauges can be placed on the load bearing internal structure of the drill between the rigid attachment and the drill burr holder. With this embodiment, a commercial drill can be retrofitted with a grounded thread fitting at its proximal end, to which an outer casing could be secured. Sensors embedded on the outside of the original casing structure would simplify and facilitate the sensor gauge placement process.

This embodiment entails bonding resistive semiconductor strain gauges on the drill structure with a thin layer of epoxy adhesive to sense three orthogonal translational under a wide range of frequencies, which are produced principally by the burr contact with the bone tissue. Semiconductor strain gauges provide very viable force sensing capabilities since their electrical resistance change to strain ratio (gauge factor) is very high. However, temperature sensors may be required to compensate for thermal strain and other non-linear properties. Also, the gauge factor of semiconductor strain gauges exhibit non-linearity over a range of forces but this behavior can be addressed with software mappings, lookup tables, curve fitting or other compensation techniques. Also, long term drift of the gauge factor may need to be addressed with periodic intervals of minor recalibration techniques. Other possible embodiments of bonding techniques include molecular bonding and diffusion. Also, foil gauges are strong candidates but they tend to have a low gauge factor.

A six DOF strain gauge arrangement, which is based on a well-published and proven Wheatstone bridge circuit design techniques, can be realized. The six-DOF configuration provides drill moment forces, which affords the possibility for the controller to recognize and respond to signature force and moment patterns induced by drill walking patterns. Non-walking cutting scenarios generate a moment, which is perpendicular to (both) the direction of the translational force and the axial direction of the drill bit and is proportional to the translational contact force and the drill bit length. A walking cutter does not generate this moment vector signature.

Previous and current position states of the burr and the current and previous contact force states of the burr, high resolution material properties of the bone such as hardness and the cutting characteristics of the burr provide robust state data to determine nearly optimal drill rotational speed to remove bone material while protecting against the possibility of injuring restricted osseous regions with a rotating drill, within reasonable surgical conduct and behavior. Specifically, the derivation of the optimal drill RPM is based on the shortest anticipated time that the cutting burr can reach an injurious region and the deceleration factor of the drill motor (or control system such as an external brake) to stop the rotation of the burr for the given material removal environment.

Equation 1 formulates a theoretical optimal RPM as follows:

Optimal RPM=Drill burr intrusion time*RPM deceleration factor; (1)

Note: The RPM deceleration factor is influenced by cutting resistance of the environment, drill motor capacity and control techniques.

In order for the controller to protect injurious regions that are completely exposed (i.e. no residual osseous material protecting the injurious region from the drill burr), the drill RPM will be set to a low value since the drill can be inadvertently accelerated quickly by the surgeon. Predictive trajectory models can be constructed to estimate potential intrusion times (i.e. surgeon penetrates into an injurious region). However, proper preoperative surgical planning should produce a final sheathing covering the injurious regions. The final sheathing is removed with a very low abrasive cutter in order for the controller to provide reasonable RPM commands as the injurious regions are being exposed in the final stages of the resection procedure. Also, force contact monitoring can disable the drill immediately to prevent injurious intrusions.

During most of the resection process, in which the injurious regions are enclosed with osseous coverings, the controller can employ a material removal energy and power paradigm to estimate the travel time of the burr to reach an injurious region. That is, each anticipated path of the burr within the remaining osseous material to be resected represents a specific material removal energy content and the drill state along the same path represents a material removal energy rate (i.e. power). The estimated time for the burr to reach an injurious region along a specific path is simply expressed in equation 2:

Drill burr intrusion time=material removal energy content/material removal power(i.e. energy rate) (2)

The material removal energy content is a function of the volumetric geometry (i.e. swept volume of the tool) and material properties (i.e. bone hardness, etc.) within the volumetric tool path. On the other hand, the material removal rate of the drill is a function of the contact forces of the drill, abrasiveness and shape of the burr, drill motor performance characteristics, and the RPM of the drill. In practice, this embodiment employs milling and drilling (i.e. plunging) formulas to estimate the time that a burr may potentially reach an injurious region. The controller adjusts the RPM (i.e. removal rate) such that the drill motor (or external brake) can stop the burr rotation before the surgeon has the opportunity to penetrate the surface boundary outside of the targeted anatomical cavity.

For example, the resulting feed rate (i.e. plunge or milling rate) for a drilling operation with a spherical burr as a function of applied axial or lateral force, cutter diameter, cutter abrasiveness, material hardness and RPM is expressed in equation 3: (Note: More sophisticated feed rate relationships can be substituted into this embodiment).

FR=AF·RPM·BA/(MH·BD); (3)

where
FR=Feed rate of the burr;
AF=Applied force (i.e. force applied by surgeon);
RPM=Rotational rate of the cutting burr (i.e. Revolution per Minute);
BA=Burr abrasiveness (in the cutting direction);
MH=Material hardness from collection bone density within swept cutter path;
BD=Burr diameter;
Equation 3 indicates that the feed rate of the burr is directly proportional to the force applied by the surgeon. The burr diameter will be accurately known. The effective material hardness of the bone can be derived from the volumetric distribution of the material hardness of the voxels, which are enclosed by the swept volume of cutter path. Again, the well established Voxmap-Pointshell algorithm, which facilitates collision and intersection detection and proximity estimations for voxel based models, could be employed to capture and collect the voxel data as the tool cutter simulates approaching an injurious surface along a candidate cutter path. A weighted average of the product the voxel volume and material hardness can serve as the effective material hardness. Essentially, this technique produces an effective material hardness value, which represents a consistent scale factor of the energy removal content of the cutter path. The burr abrasiveness factor will be calibrated prior to the surgical procedure and optionally will be part of the preoperative tool registration process. The resulting optimal RPM will provide sufficient time to permit the controller to stop the drill rotation before an injurious region is penetrated. The inclusion of force permits optimal RPM to be employed particularly in the situations where the remaining layers of resectable material are sufficiently thin. A surgeon that applies a light force will be rewarded with higher RPM output of the drill. Without feedback of the contact force of the drill, a prior art controller would need to assume a worst case scenario for the applied force on thin layers of remaining material and subsequently command a conservatively low and inherently inefficient RPM value. Moreover, the correlation of the sampling history of the operating drill parameters such as drill RPM, contact force, drill path, bone density etc., with the measured removal rates will provide excellent means to dynamically fine-tune the predictive removal rate models during the surgical process and monitor the wear and usage time of the cutting burr.

The theoretical optimal RPM of the cutting burr to remove osseous tissue along an anticipated tool path to a surface boundary of the targeted shape of the workpiece produces a travel time that matches the corresponding deceleration time of the drill motor as expressed in equation 4.

TTT=DDT; (4)

where
TTT=Total travel time of the tool (drill) path;
DDT=Drill (RPM) deceleration time;
Substituting expressions for the deceleration time (DDT)

TTT=TPL/FR; (5)

where

### TPL=Total Path Length;

FR=Feed rate of burr;
Substituting expressions for the deceleration time (DDT) (i.e. TTT)

DDT=RPM/RDR; (6)

where
RPM=RPM of the drill;

### RDR=RPM Deceleration Rate;

Equating total travel time with RPM deceleration time yields equation 7

TPL/FR=RPM/RDR; (7)

Let

Beta=Material Hardness·Burr Diameter·RPM Deceleration Rate/Burr Abrasiveness;

AF=Force applied by the surgeon;
and substituting the expression for feed rate (FR) in equation 3 into equation 7 yields equation 8 after simplification.

RPM²=TPL·Beta/AF; (8)

Solving for the theoretical optimal RPM produces equation 9.

RPM=(TPL·Beta/AF)^{1/2}; (9)

The Beta value will include a safety factor in order to produce an RPM with a reasonable safety margin. Equation 9 reflects the approach to derive an efficient RPM for a spherically shaped cutting burr. The burr calibration technique, which is explained later, provides a layer of abstraction to determine the value for Beta for a specific burr without regard to its shape.

Although, almost invariably a very low abrasive burr is employed during the process of removing the final layer, the varying contact force of the drill and the inconsistent thicknesses of the remaining layers during the final stages of the milling process may vacillate the RPM output to some degree. In response, the controller may employ predictive models of the burr's path of travel and subsequently mitigate the RPM variations in a safe manner by forecasting the underlying depths of the thin surfaces lying in the projected path of the burr. However, the standard operating procedure of utilizing a low abrasive cutter during the final stage of the resection process should dramatically attenuate RPM dithering.

One of the more challenging tasks for the otologic surgeon will be attaining the proper depth of the anatomical cavity. One variant embodiment minimizes RPM dithering and assists the surgeon with achieving high resolution depth cuts by incorporating a linear spring with an adjustable stiffness attached between the drill base and the distal end of the ACMM such that the axial direction of the drill is compliant. The stiffness can be adjusted to a relatively weak value, which enables the surgeon to “float” and “ride” the burr over the final osseous layers. The nearly consistent force of the spring and the nearly optimal cutting RPM of the drill will smoothly advance the cutting depth of the burr. For example, a soft spring stiffness of five lb/in would permit a less experienced surgeon to maintain a reasonably consistent axial contact force since gross plunging adjustments have little effect on the burr contact force. The additional axial motion of the drill may need to be sensed with an appropriate feedback device. The compliance of the spring will afford the surgeon the opportunity to concentrate on keeping the burr within the resectable region and to permit the spring force to naturally advance the depth of the cut. The drill RPM will be stopped when the burr reaches a surface boundary and the relatively small residual spring force will not be able to penetrate the contacting bone tissue. The attachment piece 23 in FIG. 2 to secure the exterior shell of the drill handle could be constructed to provide axial compliance. The handle casing would be axially compliant relative to the drill. This scheme would not require that the compliance offset be measured by a position feedback sensor.

This axially compliant approach must be optionally engaged during the surgical process since a low stiffness spring acts as a low pass filter for haptic feedback and may mask tactile sensations, which may be needed to assist the surgeon with additional sensory cues. Also, for egg shell injurious regions, if the drill burr pierces the shell there is no active mechanism to prevent the spring from pushing the burr into the soft tissue region of the egg-like structure.

The cutting edges of the burr wear over time and tend to lose their abrasiveness or cutting capacity. Moreover, it may be problematic to quantify the abrasiveness of a burr in the proper context of the material removal rate formulas internally implemented in the controller. The force sensing capability will permit the surgeon to determine the abrasiveness of the surgical burr by following a simple preoperative calibration procedure. The procedure will dictate that the surgeon drill an artifact, which has a known material hardness, in both the vertical and horizontal directions. An accurate value of the abrasiveness of the burr will be able to be derived from the contact force readings, the rate of tool travel, the drill RPM, and the material hardness of the artifact.

One embodiment leverages the proven capabilities of a temporal bone surgical simulation program with haptic feedback, in which medical residents are trained to perform bone dissection procedures on realistic, complex and detailed anatomical models generated by imaging scans of physical specimens. A simulator must continuously track the motion of the joystick, which substitutes as a cutting burr, and subsequently detect cutter collisions and intersections with the residual bone tissue. Subsequently, the simulator must determine the voxels that were removed and estimate an appropriate reaction force, which is based on bone density and the shape of the burr, to relay to the haptic device.

One embodiment exploits the material removal capability via intersection and collision detection and the corresponding updated display capabilities of the surgical temporal bone simulator. Also, the bone density information in the voxels can be leveraged to determine material removal energy content contained within a tool path. The simulator program or some form of its re-locatable software components may need to be extended in the following manner to provide an image-guidance capability:
1) Enable the surgeon to generate a targeted shape of the anatomical cavity
2) Accept high frequency tool cutter position updates from the real-time controller in order to determine material removed in the virtual model
3) Determine potential tool paths to surface boundaries of the targeted anatomical cavity
4) Compute material removal energy content or effective material hardness of a tool path to the surface boundary of the targeted anatomical cavity
5) Display surgical cues such as color coded depths, material drilling times, etc. on a dedicated display monitor
6) Interface to a digital microscope to provide surgical cues

Of course, a commercially available image guided program or collection of software components that provide similar capabilities would serve as a satisfactory alternative to the extended version of the temporal bone dissection simulator.

A substantial and common portion of some of the proposed embodiments employs well published and established, image-guided surgical techniques in the areas of virtual model construction of the surgical site, calibration of the registration probe, registration of the physical target site with its virtual counterpart model, tracking the location and orientation of the surgical tool and detection of burr collision and intersection with bone tissue to recalculate the reshaped geometry of the anatomical cavity. These techniques are adopted and practiced by robot-assisted surgery and are equally applicable to a manually maneuvered surgical tool approach. An overview of the sequence of execution of these methods in the disclosed embodiments is presented below. However, these prior art practices do not consider the applied forces of the surgeon, the bone density distribution of the anticipated tool path and the material removal capacity of the cutting burr to derive an efficient drill RPM. Moreover, prior art without a force sensing capability cannot react with the appropriate RPM in a sufficiently responsive manner.

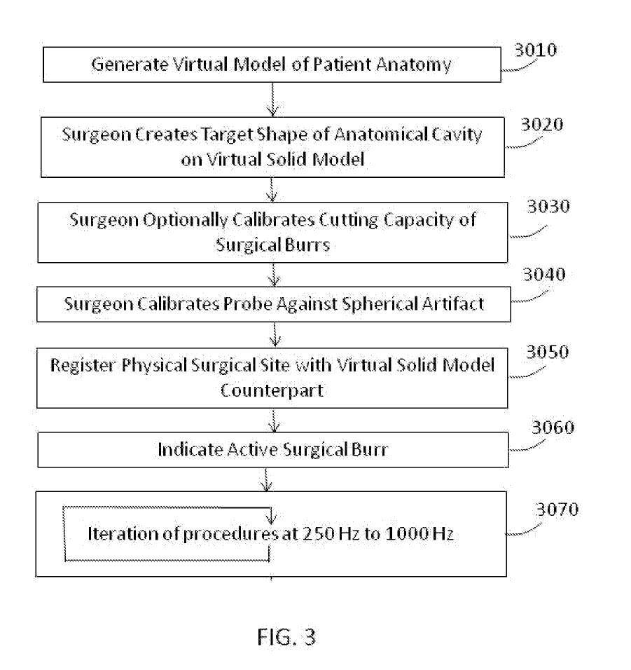

As indicated in FIG. 3, the type of image guided surgery practiced in this embodiment requires that a virtual solid model 3010 of the surgical site be generated. Precise virtual solid models of the surgical site can be generated from successive, parallel cross sectional slices of preoperative computerized axial tomography (CAT) or magnetic resonance imaging (MRI) diagnostic scans. The solid model can be represented with constructive solid geometry (CSG), boundary representations, voxels or other techniques in which volumetric data such as bone density can be associated with the model. A simulation program 3020 assists the surgeon with defining the regions of resection on the virtual solid model as part of the preoperative planning process. The region of the resected tissue defines the targeted cavity boundaries (i.e. voxels), which are safeguarded from intrusion throughout the entire surgical process.

The surgeon has the option 3030 to calibrate his surgical burrs with respect to the physical geometric parameters of the burr's shape, tool length, and cutting capacity. In this calibration procedure the surgeon touches different points of the burr on a registered metrological, spherically shaped artifact. The controller will employ force sensing to detect the burr contacting the spherical artifact. For a spherical shaped burr in which its location and orientation are accurately defined, the surgeon should touch the artifact at minimum of four times in order to enable the controller to perform an accurate least squares estimate of the location of the center of the spherical burr and its radius. A preferred technique would entail the surgeon touching the sphere on at least 16 widely different locations on the sphere and burr in order to provide a properly weighted sampling of data and an accurate derivation of the residual error of the probe location and radius through well known regression fitting techniques.

The burr cutting capacity 3030 can be calibrated by the controller by monitoring the drilling and milling processes, which are performed by the surgeon on an artifact with a known material hardness. The controller can deduce the effective axial and lateral feed rates of the burr as a function of the applied force of the surgeon and RPM of the drill by correlating the applied forces of the surgeon and the RPM of the drill with the measured feed rate of the burr. Equation 10 describes the total lateral travel distance of a tool path, in which the burr cutting capacity (i.e. feed rate) is calibrated.

$$LD = {\sum }_{n = 1}^{j} {LD}_{n} \qquad ( 10 )$$

where
LD=Total lateral distance traveled (measured and accumulated by the controller);
j=the number of samples measured by controller;
LD=individual lateral distance traveled during the n^{th} sample;

The controller on a 250 Hz or greater frequency determines and accumulates the axial and lateral motion increments of the burr and their associated applied force and RPM.

Equation 11 is derived from equation 10 by substituting the sampled lateral distances as a function of the applied force and RPM.

$$LD = FRF \cdot MH \cdot {\sum }_{n = 1}^{j} {F}_{n} \cdot {RPM}_{n} \cdot {t}_{n} \qquad ( 11 )$$

where
FRF=Feed rate factor (i.e. burr cutting capacity factor) of the burr being calibrated;
MH=Material hardness of the calibration artifact;
Fₙ=Lateral force applied by surgeon during the n^{th} sample;
RPM=RPM of the drill during the n^{th} sample;
tₙ=individual time interval at the n^{th} sample, which can be varied in order to balance the objective of filtering high frequency cutting forces with capturing its corresponding lateral distance increments;
Solving for FRF in equation 11, the feed rate of the burr, produces equation 12;

$$FRF = \frac{LD}{MH \cdot {\sum }_{n = 1}^{j} {F}_{n} \cdot {RPM}_{n} \cdot {t}_{n}} \qquad ( 12 )$$

The calibration procedure can be readily extended to derive non-linear relationships between the burr feed rate and the applied force and RPM. These relationships might be needed for extreme ends of the applied force range and the low range of the RPM. In this case during the calibration procedure, the surgeon would need to apply slowly varying forces that span the allowable force range in both the axial and lateral directions and the controller would coordinate these forces with a range of RPM commands. A lookup table or a curve fitting formula that expresses the burr feed rate as function of the applied force and RPM can be employed to address non-linearity in the relationship between burr feed rate and applied force and RPM.

A deceleration rate for the burr can be estimated accurately during the burr calibration process. The material hardness of the resected material (i.e. the removal material energy in contact with the rotating burr) will help the RPM of the burr to decelerate quickly. The calibration procedure quantifies the lateral and axial deceleration rates of the burr by commanding the burr to stop under varying applied forces and measuring the corresponding time for the burr RPM to reach zero. This deceleration data can be extrapolated for specific bone densities and applied forces of the burr during the surgical process for aggressively attaining optimal drill RPM.

A dedicated probe rather than a burr should be used to register the physical surgical site in relation to its virtual model counterpart. The probe can be calibrated against the metrological artifact in the shape of a sphere 3040 in order to establish its tip location relative to the coordinate system of the distal end of the ACCM. It is extremely important to construct an accurate coordinate transformation between the physical surgical site and its virtual model since any registration errors are reflected in the accuracy of the entire surgical process. The proper registration of the probe tip to the distal end of the ACCM represents a critical prerequisite to map accurately the surgical site to its corresponding virtual solid model.

The registration process 3050 enables the controller and simulation program to correlate the physical position of the drill to its virtual counterpart position in the virtual model. Surgical navigation and monitoring of the surgical burr requires that the digital model of the surgical site be mapped or registered to the corresponding physical space of the anatomy of the patient. In one typical registration technique 3050, the physical locations of readily identifiable anatomical feature points are correlated with their virtual model counterparts. If the bone anatomy is repositioned dynamically during surgery, then provisions can be made to track and subsequently compensate for the base motion effects of the osseous surgical region. A list of other feature points should be checked against the virtual model in order to verify the accuracy of the registration transformation.

The surgeon selects from a list of calibrated burr types 3060 in order to indicate the drill burr currently active in the surgical process. Subsequently, the controller will apply the corresponding burr location and geometry and cutting characteristics in order to track removed tissue, reconstruct the geometry of the residual cavity and command an efficient RPM. As an option, the surgeon could touch a sanitized metrological, spherical artifact at a number of locations to verify that the active burr was identified correctly.

The continuous updates 3070 of the locations and orientations of the drill burr from the real-time controller to the virtual model simulation program enables the virtual model simulation program to determine burr collision and intersection occurrences within the residual bone tissue, which in this embodiment changes the status of corresponding voxel data to a removed state and updates the display data of the digital solid model, calculates closest points of surface boundaries to the burr, provides a snap shot of the bone density surrounding the burr and re-computes the material removal energy content within potential tool paths leading to nearby surface boundaries.

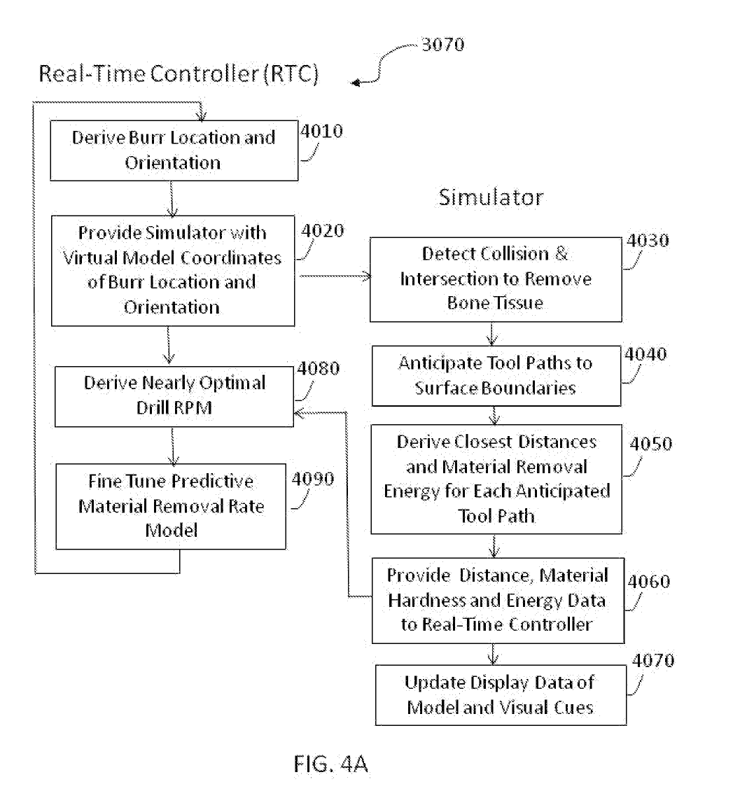

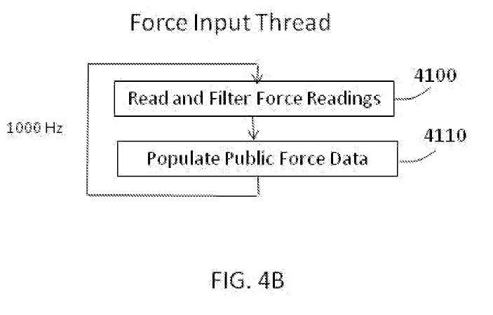

FIG. 4A displays a detailed schematic overview of the iterative methods 3070, which are executed by the controller during the resection process. The joint positions of the ACCM are input 4010 on a 250 Hz or greater basis. The forward kinematics of the joint positions of the ACCM establishes the pose (location and orientation) of the drill burr. The updated pose of the burr is relayed to the simulator on the 250 Hz or greater basis 4020. Many techniques such as real-time Ethernet connections, memory drops, USB links, etc., which interface between the real-time controller and the virtual temporal bone dissection simulator are possible and will be dictated by the software architecture of the control system. In this embodiment the temporal bone simulator may not have the CPU resources to detect collision and intersection of the burr with the residual material, derive material removal energy content of perspective tool paths, etc., on a 250 Hz or greater basis 4030. Consequently, the simulator may execute on a lower frequency cycle and manipulate the higher frequency tools updates to accommodate the lower frequency tasks.

The simulation processing determines the limiting path of the probable tool paths 4040 to reach the boundary of the targeted cavity. Part of the method will derive the points along the cavity surface that are the closest distance to the burr, which will account for possible worst case scenarios. That is, the controller must account for the possibility of the burr heading directly towards a closest boundary point. Predictive models of the burr trajectory are exploited to safeguard that the arrival time of the burr to a projected intersection point on a potentially targeted but bare (no intervening material) cavity surface. The trajectory estimation must be performed on a high frequency basis of 250 Hz or greater in order produce nearly optimal RPM. However, if the drill rotation is stopped automatically after the controller detects low contact forces have been present for sufficient time interval, then the need for the trajectory estimation process is eliminated. The material removal energy content 4050 of each prospective path to a targeted cavity surface needs to be computed until at least to the point that its travel time is guaranteed to be sufficiently greater than the RPM deceleration time.

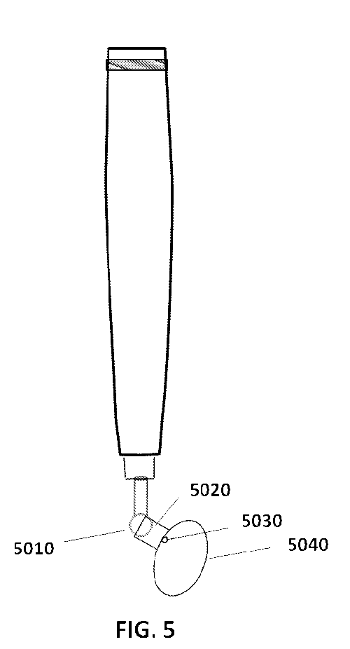

FIG. 5 illustrates a prospective cutter path 5010, whose start and end points are respectively the current location of the drill burr 5020 and the closest point 5030 of an injurious region 5040 to the drill burr. The Voxmap-Pointshell algorithm can be exploited to determine closest voxels to the drill burr. Other potential cutter paths can be considered based on the direction of the drill burr, its contact force direction, etc. The voxel data enclosed by the swept volume of the cutter path can be analyzed and weighted to determine an effective material hardness. Again, each potential path can be addressed separately and consequently analyzed on a separate processor core in order to leverage parallel processing.

The simulator updates the real-time controller with a list of path lengths with coordinates of their associated target end points and material removal energy content (or equivalent material hardness) 4060. The real-time controller will employ equation 9 to determine the optimal removal rate of the burr (i.e. optimal RPM) as a function of the applied contact force of the burr, burr cutting or feed rate capacity and material removal energy content of the potential tool paths 4080.

An aspect of this embodiment addresses the process of the controller tracking and correlating the burr feed rate with the drill RPM, material hardness in the bone removed by the burr, and the burr force applied by the surgeon 4090. The controller will be able to exercise the same techniques employed in the burr calibration procedure to dynamically fine-tune the lateral and axial cutting capacity of the burr during the surgical process.

The updated status of the virtual model, which includes a revised display of the surgical cues such as remaining bone material, distances to surfaces, depths, bone densities, energy content, etc., should be conveyed to the surgeon in an intuitive, interactive and informative manner in order to increase his situational awareness 4070. In particular, augmented reality techniques permit transparent graphical representations of these data types to be overlaid on the real-time image generated from a high resolution digital microscope to provide the surgeon with relevant data in his surgical field of view.

The simulator will need to update the display data on at least a 30 Hz cycle in order produce smooth tool motion and surgical cues 4070. Unlike current robotic-assisted surgeries, in which the surgeon views a virtual model on a monitor as he performs the resection procedure, it would be advantageous for the surgeon to be able to view the real-image of the surgical site generated by the microscope with visual cues of the targeted anatomical cavity 4070. However, if augmented reality techniques are not possible then a separately enclosed image of the updated virtual model embedded into the real-time image of the surgical site would prove to be beneficial. As a less favorable embodiment alternative, a separate monitor will display the visual cues with the updated virtual model and burr location and orientation, which will dictate that the surgeon look away from his surgical view to assess the surgical situational conveyed in the monitor.

FIG. 4B demonstrates a separate execution thread that reads and filters 4100 the contact forces of the burr on a 1000 Hz cycle. The high frequency input permits the controller to detect at a fine resolution material contact, which may help correct the removed material tracking performed by the simulator. Since the drill will operate at a high RPM and the forces exerted by the surgeon will operate at much lower frequency, low pass filtering techniques will be employed to capture the low frequency forces applied by the surgeon in a responsive manner. The filtered forces can be presented to the controller 4110 through public variables, managed memory access routines, etc.

In conclusion, these embodiments offer superior benefits over position-only feedback, which is employed in prior art in the following ways:
(1) Estimate far more precisely the drill burr intrusion times, which permits virtually optimal drill RPM rates and consequently more effective surgical throughput by employing the following techniques:
   a. Quantify burr abrasiveness (i.e. removal feed rate) via a simple preoperative calibration procedure
   b. Measure contact force to determine optimal RPM
   c. Determine material removal energy content of candidate tool path based on distribution of volumetric material hardness data
   d. Dynamically recalibrate material removal rates during surgical procedure
   e. Dynamically readjust deceleration factor based on monitoring cutting resistance (i.e. motor torque)
   f. Verify and correct material removal tracking performed by virtual simulation program
(2) React more quickly, precisely and safely to applied forces of the surgeon
(3) Detect drill walking and subsequently mitigate its effect
(4) Float cutting burr over thin osseous sheathing with compliant axial loads

Although the previous description includes much specificity, these should not be construed as limiting the scope of the potential embodiments but rather as simply providing examples of some possible embodiments. For example, a non-voxel based energy approach can be devised for woodworking or sculpturing on objects with consistent material properties. The artesian simply performs a simple procedure that correlates the removal rate characteristics of the cutter and the control can subsequently compute the optimal RPM speed. Consequently, the scope of the embodiments should be delineated by the appended claims and their legal equivalents in lieu of the previous examples.

What is claimed is:

1) A system consisting of:
a tool for removing material;
a workpiece requiring material to be removed to attain a target shape;
a device for tracking the pose of the tool;
a controller for regulating tool removal rate and for tracking material removed from the workpiece.

2) A system according to claim 1, wherein said tool is a rotating drill.

3) A system according to claim 1, wherein the regulation of said tool removal rate is based on the geometric distribution of energy removal content of the remaining material to be removed from said workpiece.

4) A system according to claim 2, wherein material removal energy content is confined within the swept volumes of prospective tool cutting paths.

5) A system according to claim 2, wherein said drill has its three degrees of freedom translational contact forces measured by said controller, whereby providing greater awareness of the state of the system and consequently more responsive control and permitting verification and correlation of the solid model with its physical counterpart.

6) A system according to claim 5, wherein said controller sufficiently reduces rotational speed of said drill if a suitable contact force is not applied, whereby providing a very responsive manner to stop the rotation of said drill while in free motion from inadvertently contacting a forbidden region or to discourage excessive contact forces from being applied.

7) A system according to claim 5, wherein the derivation of a candidate rotational speed of said drill is predicated upon energy removal formulations that rely on cutting and operating characteristics of said drill, applied contact forces of said drill, and energy removal content of prospective tool cutting path, whereby permitting said drill to operate at optimal rotational speed within the prospective tool cutting path.

8) A system according to claim 7, wherein said energy removal formulations are employed to derive said candidate rotational speed of said drill for generating a specific travel time of said drill along said prospective tool cutting path, whereby permitting said drill to operate at optimal rotational speed within said prospective tool cutting path.

9) A system according to claim 8, wherein said controller provides command signal to generate the lowest rotational speed from the candidate list of said prospective tool removal paths, whereby permitting said drill to operate at optimal rotational speed without intruding into forbidden regions.

10) A system according to claim 5, wherein the burr cutting characteristics of said drill is empirically derived by correlating measured energy removal parameters, whereby accurately registering the material removal capacity of the burr.

11) A system according to claim 5, wherein the burr cutting characteristics of said drill is empirically monitored and adjusted during the material removal process by correlating measured energy removal parameters, whereby updating the energy removal techniques with more accurate material removal properties of the burr.

12) A system according to claim 3, wherein said controller employs energy removal principles to provide a command signal to generate a removal material time that equals the power down time of the material removal tool, whereby permitting optimal removal rates without interfering with the forbidden region.

13) A system according to claim 2, wherein six degrees of freedom contact forces of said drill are sensed.

14) A system according to claim 13, wherein force and moment signatures of said drill are detected, whereby enabling said controller to recognize the phenomenon of drill walking and mitigate its effects by adjusting the rotational speed of said drill.

15) A system according to claim 2, wherein an outer casing of the tool handle is retrofitted onto the original housing of said drill and strain gauges are embedded in the load bearing structure of said drill, whereby eliminating the need to locate strain gauges between the burr of the said drill and the grasping points of the handle of said drill.

16) A system according to claim 2, wherein a passive axial compliance along the direction of the drill bit shaft is incorporated, whereby high frequency contact forces are mitigated and intended contact forces can be maintained more readily.

17) A system according to claim 5, wherein said translational force sensing confirms voxel content of virtual solid model, whereby affording the opportunity to correlate and adjust the voxel model with the physical model.

18) A system according to claim 5, wherein additional moment force sensing is incorporated, whereby affording the opportunity to confirm the calibrated length of the cutting tool and that the burr rather than the drill bit is making contact with the material.

19) A system according to claim 1, where said device for tracking is a kinematically redundant articulated coordinate measurement system, whereby permitting the latitude to configure the pose of the kinematically redundant articulated coordinate measurement system to minimize obstruction and adjust apparent tool inertia.

20) A system according to claim 1, where said kinematically redundant articulated coordinate measurement system has adjustable counterbalances, whereby enabling the tool to approach a weightless state during operation.
